(19) United States
(12) Patent Application Publication
(10) Pub. No.: US 2007/0248566 A1
(43) Pub. Date: Oct. 25, 2007

(54) Antimicrobial polyisocyanate and derivatives thereof

(75) Inventors: Chien-Ming Chen, Hsinchu County (TW); Chih-Kai Chiu, Taipei County (TW)

Correspondence Address: BIRCH STEWART KOLASCH & BIRCH, PO BOX 747, FALLS CHURCH, VA, 22040-0747, US

(73) Assignee: HEADWAY ADVANCED MATERIALS INC.

(21) Appl. No.: 11/409,989

(22) Filed: Apr. 25, 2006

(51) Int. Cl. A61K 31/787; A61K 31/785; C08L 75/06; C08L 75/08

(52) U.S. Cl. 424/78.27; 424/78.3; 525/438; 525/440.04; 525/440.06

(57) ABSTRACT

The present invention discloses an antimicrobial polyisocyanate and derivatives thereof, wherein a quaternary amine is added into the polymerization reaction of a polyol and a NCO-containing isocyanate/poly-isocyanate to form the antimicrobial polyisocyanate; then, the antimicrobial polyisocyanate is added into a functional resin to form an antiseptic material. As the bactericide (the quaternary amine) becomes an inseparable portion of the polyisocyanate and the antiseptic material containing the poly-isocyanic ester, the bactericide will be uniformly distributed on the surface of material; further, the bactericide will not be released out but will be maintained permanently. Such a mechanical-contact type antiseptic method can achieve a safe, persistent and environment-friendly antiseptic effect.

### FIELD OF THE INVENTION

The present invention relates to an antimicrobial polyisocyanate and derivatives thereof, particularly to an antiseptic polyisocyanate with a terminal —NCO functional group and the derivatives thereof, wherein the bactericide is an inseparable portion of the antimicrobial polyisocyanates or the derivatives thereof, therefore, the bactericide will not be washed away, and the antiseptic effect will not decay with time.

### BACKGROUND OF THE INVENTION

Microorganisms usually refer to unicellular creatures, including: coccus, bacillus, trichobacteria, and spirillum. In fact, besides various pathogens and bacteria, microorganisms also include: protozoa, which are larger than bacteria, and viruses, which are smaller than bacteria and also called intercellular bacteria. Microorganisms correlate closely with circulation, digestion, and metabolism of human bodies and also correlate closely with the fabrication of wine, soy, and vinegar. Microorganisms are indispensable for the industries of alcohol, butanol, acetone, lactic acid, antibiotics, and medicine. Microorganisms also have a very important function in evolution and environment protection. However, among over two hundred thousand or five hundred thousand microorganisms, those harmful to human beings are much more than those benefiting human beings. At present, none medicine that is universally effective to all microorganisms has been found. For the development of an antibiotic medicine, toxicity, carcinogenicity, the effects on gene, and the acid resistance of bacteria are all the critical problems.

With the evolution of society, the demand for heal care grows, and the market of the derivative products of antiseptic materials also expands rapidly. The derivative products of antiseptic materials refer to the products that an antiseptic material is added into. Before, those products were usually daily household commodities and electric appliances. Recently, the antiseptic products have expanded to building/decoration materials, plastics, rubbers, coating materials, resins, food packaging materials, fabric products, and even the high-tech and high-added-value products, such as medical equipments and materials.

Adding an antiseptic material into a product is to provide the surface of the product with an antiseptic function so that microorganisms will not grow on the surface of the product lest diseases spread. Thereby, the hygienic objective can be achieved.

However, it is well known that the development of a safe, long-acting, environment-friendly antiseptic material is a hard problem for the fields of disinfection and public health and has none satisfactory solution yet. Thus, the antiseptic material having a direct and persistent disinfection effect on various germs likely to contact daily is an objective the researchers desirous to achieve.

The current antiseptic technologies include: antibiotics, quaternary amines, and inorganic disinfectants (such as iodine, silver salts, copper salts, and zinc salts). The operation method thereof is that the abovementioned antiseptic agents are slowly released into the ambient solution to kill the germs in the solution.

Antiseptic agents and antiseptic materials may the following two disinfection mechanisms:
(a) The antiseptic agent entering into the cells of germs interferes with the gene reproduction, and thus, the objective of disinfection is achieved. Herein, silver salts are used to exemplify the mechanism of inorganic disinfectants. Among inorganic disinfectants, silver salts are not so cheap as but more effective than copper salts. When germs contact an inorganic silver salt, the silver ions will pass through the cell membranes of germs and enter into the cytoplasm of the germ cells. The silver ion will replace the hydrogen of the sulfhydryl group on the RNA and DNA of germ cell to form a silver-sulfur group. Thereby, the germs cannot reproduce their genes, and the objective of disinfection is achieved. The mechanism of small-molecule organic antiseptic agents also functions similarly. Molecules of a small-molecule organic antiseptic agent will also pass through the cell membranes of germs and enter into the cytoplasm of the germ cells, and the metabolism of the germs will be inhibited. Thereby, the germs cannot reproduce their genes, and the objective of disinfection is achieved.
(b) The positively-charged functional groups of a quaternary amine will interact with the surfaces of germ cells; thus, the cell membranes will be destroyed, or the metabolism of cytoplasm will be interfered with. Thereby, the germs will either die or be unable to reproduce their genes. The active portion of a quaternary amine is its positively-charged functional groups. The positively-charged functional groups will induce negative charges on the surfaces of germs. The imbalanced charge distribution further induces abnormal metabolism of germ cells. Thereby, the germs cannot reproduce their genes, and the objective of disinfection is achieved.

There is also a theory pointing out: owing to the similarity in molecular structures, the quaternary amines and the phospholipids—a constituent of germ membrane—may interact; thus, the membranes of germs are destroyed, which causes the exposure of cytoplasm and the death of germs. Thereby, the objective of disinfection is achieved.

In comparison with the slow-release type antiseptic agents described in (a), the antiseptic agents described in (b) may be regarded as mechanical-contact type. The slow-release type antiseptic agents described in (a) have to pass through the cell membranes of germs and enter into the cytoplasm of germs, so that the metabolism of germs will be interfered with, the gene reproduction is inhibited, and the objective of disinfection is achieved. However, it is unnecessary for the mechanical-contact type antiseptic agents described in (b) to interact with the cytoplasm. Therefore, the drug resistance induced by germ mutation is unlikely to occur.

Besides, the antiseptic effect of common antiseptic products will gradually decay with time because the antiseptic agent is not a built-in and inseparable portion synthesized with the molecules of the antiseptic products but a component ultra added into those antiseptic products. Therefore, the antiseptic agent will be washed away and finally ineffective.

### SUMMARY OF THE INVENTION

The primary objective of the present invention is to combine a quaternary-amine-group bactericide and related materials to form a safe, long-acting and environment-friendly polyisocyanate adducts with terminal —NCO functional groups and derivatives thereof, which can be used as the hardener of two-component polyurethane coatings and adhesives, and can also be used as a component moisture curing polyurethane. The polyisocyanate of the present invention can be uniformly distributed on the surface of materials to achieve a safe, persistent, and environment-friendly antiseptic effect.

To achieve the abovementioned objectives, a reactive quaternary-amine bactericide is adopted to participate in the reaction with a polyisocyanate form an inseparable portion in the chemical structure of this polyisocyanate adduct. As the quaternary-amine bactericide has become an inseparable portion of the polyisocyanate structure, the quaternary amine will not be released out or washed away when the antiseptic polyisocyanate is used in one-component or two-component resins. Therefore, the present invention can achieve a safe, persistent, and environment-friendly antiseptic effect.

### DETAILED DESCRIPTION OF THE INVENTION

The technical contents of the present invention will be described in detail below.

In the present invention, a polyol and one of the isocyanate group containing chemicals, such as diisocyanate, triisocyanate, or polyisocyanate are reacted into urethane prepolymer with terminal —NCO functional groups, wherein a reactive quaternary anine is added into the reaction with the NCO group then chemically bound to the molecular structure of the polyisocyanate adducts. This quaternary amine becomes the inseparable portion of the polyisocyanate adducts to provide the antiseptic function.

The antimicrobial polyisocyanates can be used to react with the resins containing —OH group, —NH₂ group or —SH group, even used alone to react with H₂O moisture to form the antiseptic material.

The reaction formula of the polymerization of the antimicrobial polyisocyanates is expressed as follows: 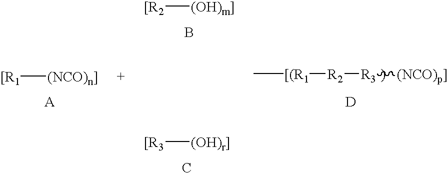
wherein p=n−m−r>0. The reactants of the reaction formula are described below.

Reactant A is an NCO-containing isocyanate or an NCO-containing polyisocyanate, and may be selected from the group consisting of:
1. Aliphatic isocyanate, such as 1,6-hexamethylene diisocyanate and Isophorone Diisocyanate;
2. Aromatic isocyanate, such as 2,4-toluene diisocyanate and Methylene diphenyl diisocyanate; and
3. Poly-isocyanate, such as HDI trimer, HDI dimmer, and TDI trimer.

Reactant B is a polyol, and may be selected from the group consisting of
1. Low molecular weight polyols, such as ethylene Glycol, 1,4-butanediol, and trimethylol propane;
2. High molecular weight polyols, such as polyester polyol and polyether polyol.

Reactant C is a quaternary-amine bactericide containing reactive functional groups, and may be selected from the group consisting of: 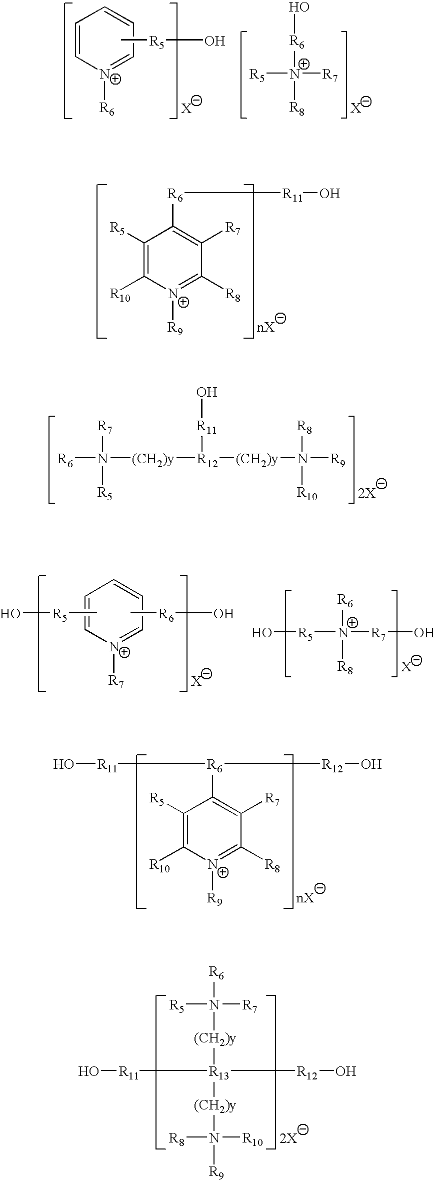
wherein n is an integer within the range from 1 to 1000; y is an integer within the range from 1 to 10 and preferably within the range from 1 to 3; X is a halogen element selected from the group consisting of fluorine, chlorine, bromine, iodine, and astatine; and the OH functional group can be replaced with an NH₂ functional group, and
wherein R1˜R8 in the abovementioned quaternary amines are elements or functional groups selected from the group consisting of: hydrogen, alkyl, alkenyl, alkynyl, acyl, aryl, carboxylate, alkoxycarbonyl, carboxamido, alkylamino, acylamino, alkoxyl, acyloxy, hydroxyalkyl, alkoxyalkyl, aminoalkyl alkylamino, thio, alkylthio, thioalkyl, alkylthio, carbamoyl, urea, thiourea, sulfonyl, sulfonate, sulfonamide, sulfonylamino, and sulfonyloxy.

The abovementioned antimicrobial polyisocyanates can be added into a functional resin to form an antiseptic material, and the reaction formula is expressed by: 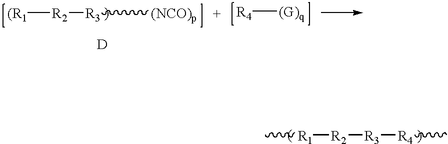
Wherein the number of the reactive groups of the antiseptic poly-isocyanic ester P□ the number of the reactive groups of the functional resin q; the reactive group of the functional resin is selected from the group consisting of alkyd resin, polyester, hydroxyl functional epoxy resin, polycaprolactone, polycarbonate, polyether, poly amine, poly carbohydrate, and hydroxyl cellulose.

The present invention will be further numerically exemplified below.

The synthesis of the poly-isocyanic ester with terminal —NCO functional groups of the present invention is described below.

The reactants occupy 70% the total volume of the raw materials, and the solvents occupy 30% the total volume of the raw materials. The reactants include: aliphatic poly-isocyanate with NCO %=23% and dodecyl (2-hydroxyethyl)dimethylammonium bromide with OH value=305; the proportion by weight of aliphatic poly-isocyanate to dodecyl (2-hydroxyethyl)dimethylammonium bromide is 77.78:22.22. The solvent consists of BAC and Dimethylacetamide, and the proportion by weight of BAC to Dimethylacetamide is 3/1. The raw materials further comprise: 1000 ppm anti-oxidant and 600 ppm catalyst. The reaction of the abovementioned raw materials is undertaken at a temperature within the range from 80° to 90° for 12 hours. The reaction products comprise an antiseptic poly-isocyanic ester with NCO %=7.0˜7.5%.

The sterilizing effect of the abovementioned poly-isocyanic ester with —NCO functional groups is analyzed as follows.

The abovementioned polyisocyanates with terminal —NCO functional groups functioning as hardener is mixed with acrylic polyol, and the mixture is processed and applied onto the surface a PP plastic. The sample is tested with JIS Z 2801:2000 a film-test method for antiseptic products to verify the sterilizing effect of the abovementioned polyisocyanates with terminal —NCO functional groups. The test result shows that the present invention has a superior sterilizing effect, and the bactericidal coefficient thereof can reach as high as 99.9%.

| Test bacterium | Sterilizing effect |
|---|---|
| Staphylococcus auresus | Sterilizing value = 2.3 |
| (AATCC-6538P) | (Bactericidal coefficient [symbol] 99.9%) |
| Escherichia coli | Sterilizing value = 2.3 |
| (AATCC-8739) | (Bactericidal coefficient [symbol] 99.9%) |

Thus, the product, which integrates acrylic polyol and the poly-isocyanic ester with terminal —NCO functional groups of present invention, can provide the resin material with a persistent antiseptic effect. It results from that the reactive quaternary-amine bactericide participates in the synthesis of the polyisocyanates and becomes an inseparable portion of the polyisocyanates. As bactericide becomes an inseparable portion of the polyisocyanates, the bactericide will not be washed away, and the antiseptic effect will be maintained permanently.

The disinfection mechanisms of common inorganic disinfectants are usually of dissolution type or slow-release type, and the antiseptic effect thereof will gradually diminish by frequent washing. The mechanical-contact type disinfection mechanism of the present invention is different from those of common inorganic disinfectants. In the present invention, the quaternary-amine bactericide is an inseparable portion of the polyisocyanates; thus, the bactericide will not be released out but can be uniformly distributed on the surface of material. Therefore, the present invention has a safe, persistent and environment-friendly antiseptic effect. The polyisocyanates of the present invention can be used as the hardener of the polyurethane of a two-component resin, the crosslinker of a two-component adhesive, the pre-polymer of a polyurethane elastomer, the pre-polymer of a foamed the polyisocyanates, and the pre-polymer of a moisture curing polyurethane.

Those described above are only the preferred embodiments of the present invention; however, it is not intended to limit the scope of the present invention, and any equivalent modification and variation according to the spirit of the present invention is to be included within the scope of the present invention. For example, the pre-polymers and hardeners of polyurethane, which are the reaction products of any one of the commonly used mono-isocyanate and poly-isocyanates and any one of the quaternary amines with at least one OH group, are to be included within the scope of the claims of the present invention.

What is claimed is:

1. An antimicrobial polyisocyanate, formed via adding a quaternary amine into the polymerization reaction of a polyol and an NCO-containing isocyanate or an NCO-containing poly-isocyanate to undertake a reaction according to the following reaction formula: 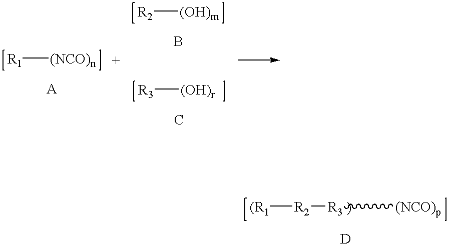
wherein p=n−m−r>0; A is an NCO-containing isocyanate or an NCO-containing poly-isocyanate; B is a polyol; C is a quaternary-amine bactericide; and D is an antiseptic poly-isocyanic ester.

2. The antimicrobial polyisocyanate according to claim 1, wherein said NCO-containing isocyanate or said NCO-containing poly-isocyanate is selected from the group consisting of aliphatic isocyanate, aromatic isocyanate, and poly-isocyanate.

3. The antimicrobial polyisocyanate according to claim 1, wherein said polyol may be a low-molecular-weight polyol selected from the group consisting of ethylene glycol, 1,4-butanediol, and trimethylol propane; or a high-molecular-weight polyol selected from the group consisting of polyester polyol and polyether polyol.

4. The antimicrobial polyisocyanate according to claim 1, wherein said quaternary amine is selected from the group consisting of

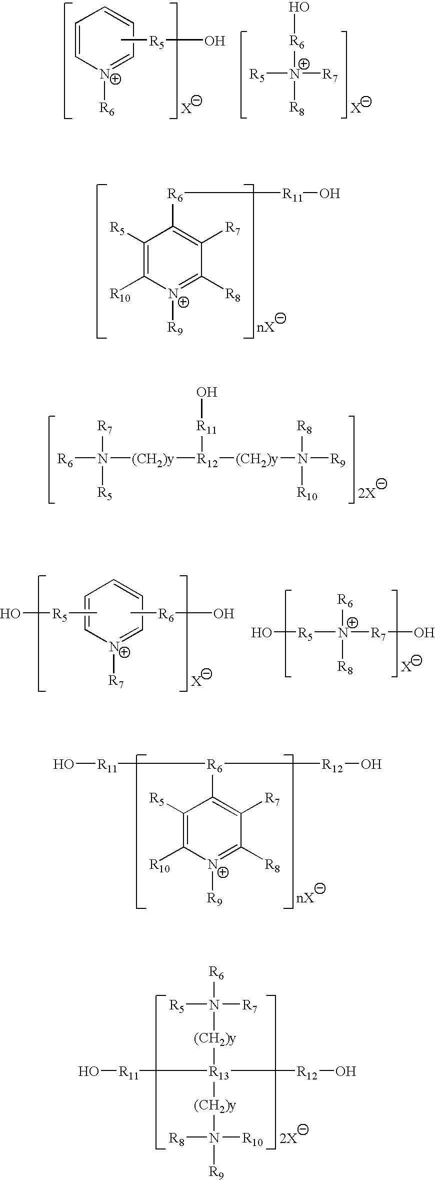

5. The antimicrobial polyisocyanate according to claim 4, wherein each of from R1 to R8 in said quaternary amines is an elements or a functional group selected from the group consisting of: hydrogen, alkyl, alkenyl, alkynyl, acyl, aryl, carboxylate, alkoxycarbonyl, carboxamido, alkylamino, acylamino, alkoxyl, acyloxy, hydroxyalkyl, alkoxyalkyl, aminoalkyl □ alkylamino, thio, alkylthio, thioalkyl, alkylthio, carbamoyl, urea, thiourea, sulfonyl, sulfonate, sulfonamide, sulfonylamino, and sulfonyloxy.

6. The antimicrobial polyisocyanate according to claim 4, wherein X is a halogen element selected from the group consisting of fluorine, chlorine, bromine, iodine, and astatine.

7. The antimicrobial polyisocyanate according to claim 4, wherein n is within the range of from 1 to 1000.

8. The antimicrobial polyisocyanate according to claim 4, wherein y is within the range of from 1 to 10 and preferably within the range of from 1 to 3.

9. The antimicrobial polyisocyanate according to claim 4, comprising PU (polyurethane) pre-polymers, poly-isocyanic esters, and the derivatives of said poly-isocyanic esters, which are the reaction products of any one of the commonly used mono-isocyanate and poly-isocyanates and any one of the quaternary amines with at least one OH group.

10. The antimicrobial polyisocyanate according to claim 4, wherein the OH group of said quaternary amine may be replaced by an amino(NH₂) group.

11. The antimicrobial polyisocyanate according to claim 1, wherein said antimicrobial polyisocyanates is added into a functional resin to form an antiseptic material, and the reaction formula is expressed by: 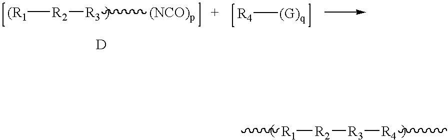
wherein the number of the reactive groups of said antimicrobial polyisocyanates P□ the number of the reactive groups of said functional resin q.

12. The antimicrobial polyisocyanate according to claim 11, wherein said NCO-containing isocyanate or said NCO-containing poly-isocyanate is selected from the group consisting of aliphatic isocyanate, aromatic isocyanate, and poly-isocyanate.

13. The antimicrobial polyisocyanate according to claim 11, wherein said polyol may be a low-molecular-weight polyol selected from the group consisting of ethylene glycol, 1,4-butanediol, and trimethylol propane; or a high-molecular-weight polyol selected from the group consisting of polyester polyol and polyether polyol.

14. The antimicrobial polyisocyanate according to claim 11, wherein said quaternary amine is selected from the group consisting of

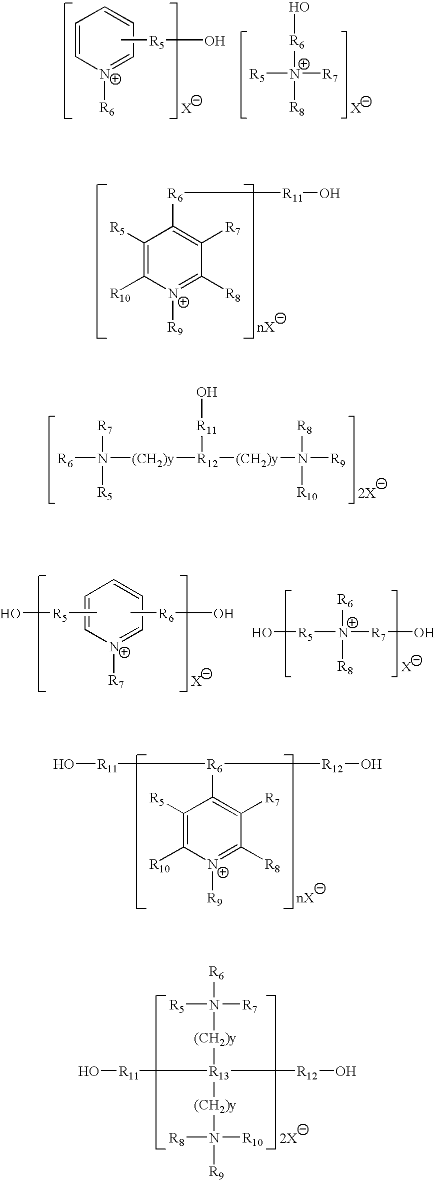

15. The antimicrobial polyisocyanate according to claim 14, wherein each of from R1 to R8 in said quaternary amines is an element or a functional group selected from the group consisting of: hydrogen, alkyl, alkenyl, alkynyl, acyl, aryl, carboxylate, alkoxycarbonyl, carboxamido, alkylamino, acylamino, alkoxyl, acyloxy, hydroxyalkyl, alkoxyalkyl, aminoalkyl □ alkylamino, thio, alkylthio, thioalkyl, alkylthio, carbamoyl, urea, thiourea, sulfonyl, sulfonate, sulfonamide, sulfonylamino, and sulfonyloxy.

16. The antimicrobial polyisocyanate according to claim 14, wherein X is a halogen element selected from the group consisting of fluorine, chlorine, bromine, iodine, and astatine.

17. The antimicrobial polyisocyanate according to claim 14, wherein n is within the range from 1 to 1000.

18. The antimicrobial polyisocyanate according to claim 14, wherein y is within the range from 1 to 10 and preferably within the range of from 1 to 3.

19. The antimicrobial polyisocyanate according to claim 14, comprising PU (polyurethane) pre-polymers, poly-isocyanic esters, and the derivatives of said poly-isocyanic esters, which are the reaction products of any one of the commonly used mono-isocyanate and poly-isocyanates and any one of the quaternary amines with at least one OH group.

20. The antimicrobial polyisocyanate according to claim 14, wherein the OH group of said quaternary amine may be replaced by an amino(NH₂) group.

21. The antimicrobial polyisocyanate according to claim 11, wherein the reactive group of said functional resin is selected from the group consisting of alkyd resin, polyester, hydroxyl functional epoxy resin, polycaprolactone, polycarbonate, polyether, poly amine, poly carbohydrate, and hydroxyl cellulose.
